(19) United States
(12) Patent Application Publication
(10) Pub. No.: US 2015/0204885 A1
(43) Pub. Date: Jul. 23, 2015

(54) METHOD FOR THE DIRECT DETECTION OF MYCOBACTERIUM TUBERCULOSIS

(71) Applicants: Nicolas POULAKIS, Athens (GB); Aggelos GRITZAPIS, Athens (GR); Vassilios TSILIVAKOS, Athens (GR)

(72) Inventors: Nicolaos Poulakis, Cholargos (GR); Aggelos Gritzapis, Cholargos (GR); Vassilios Tsilivakos, Cholargos (GR)

(21) Appl. No.: 14/420,355

(22) PCT Filed: Aug. 8, 2013
(86) PCT No.: PCT/GR2013/000043

§ 371 Date: Feb. 7, 2015

(30) Foreign Application Priority Data
Aug. 8, 2012 (GR) 20120100415

(51) Int. Cl. G01N 33/68

(52) U.S. Cl. CPC G01N 33/6854

(57) ABSTRACT

A method and kit for providing high accuracy intracellular detection of Mycobacterium tuberculosis, an infection causing either active or latent disease. The technique involves immunofluoresence in combination with flow cytometry on samples from all biological fluids and cell suspensions.

### TECHNICAL FIELD OF THE INVENTION

The present invention describes a method for the direct detection of Mycobacterium tuberculosis (hereafter, referred to as M.TB.) infection, inside phagocytes collected from peripheral blood, sputum, bronchoalveolar lavage, serositis exudates and, in general, from every biological fluid or tissue sample accrued from the human body. The said detection involves immunofluoresence in combination with flow cytometry.

### PRIOR ART

Tuberculosis is a modern plague of humanity. Globally, a total of 13.7 million prevalent TB cases were recorded in 2007, with an estimated 1.37 million of incident TB cases in 2007 coinfected with HIV. This corresponds to a prevalence of 206 cases per 100,000 that resulted in 1.756 million deaths (including 456,000 TB patients coinfected with HIV) worldwide. 500,000 cases of multidrug-resistant TB (MDRTB) defined as infection with M. tuberculosis strains resistant to at least two of the most important first-line drugs, rifampin and isoniazid) occurred in 2007 [1].

By the end of 2008, multiple drug-resistant TB (XDR-TB), defined as MDRTB strains additionally resistant to a fluoroquinolone and an injectable agent such as kanamycin, amikacin, viomycin, or capreomycin, has been found in 55 countries and territories of the world.(1,2) While MDR-TB is difficult and expensive to treat, XDR-TB is virtually an untreatable disease in most developing countries.

The World Health Organization (WHO) has estimated that one-third of the total world population is latently infected with M. tuberculosis (hereafter, referred as LTB) and that 5%-10% of infected individuals will develop active TB disease during their life time. However, the risk of developing active disease is 5%-15% every year and lifetime risk is 50% in HIV coinfected individuals. Most of the active disease cases in low TB incidence countries arise from this pool of latently infected individuals [1, 2].

The number of LTBI individuals is anticipated to rise, especially in developed countries, due to the increased number of medically immunocompromised patients, such as organ transplant recipients and cancer patients, as well as patients on treatment with biological agents (anti-TNF-a agents, etc.). In addition, a large number of refugees from countries with high inidences of TB, presumably will emigrate due to political and economic problems.

Primary infection with M.Tb. results in a disease state in only 10% of infected individuals. The rest of the infected population develops an induced immunological response, thereby averting further spread of M.Tb. which is confined to cellular complexes called tubercles. M.TB. cannot multiply in tubercles and as a result remains latent. The host, therefore, remains healthy although the pathogen could potentially revert to its virulent state, especially when the host is under reduced immunosurveilance, i.e. in the case of HIV infection.

The currently known tests for diagnosis of LTBI are based exclusively on immunological tests assesing host-specific cellular immunity to MTBC antigens. The intradermal skin test (PPD—in use for over 100 years) and the recently introduced immunity-based interferon-gamma (IFN-γ) release assays (IGRAs) that detect T cell responses following stimulation by M. tuberculosis-specific antigens, are the main tests for LTBI [3].IGRA assays include QuantiFERON-TB Gold, QuantiFERON-TB Gold In-Tube (QFT-G-IT), and T-SPOT.TB In a recent meta-analysis, the pooled sensitivity of IGRAs was 70%-90% and the pooled specificity was 93%-99%. In blood and extrasanguinous fluids, the pooled sensitivity for the diagnosis of active TB was 80% and 48%, respectively, for QFT-G-IT. The respective sensitivity for T-SPOT was 81% and 88%. In blood and extrasanguinous fluids, the pooled specificity was 79% and 82% for QFT-G-IT, and 59% and 82% for T-SPOT.TB, respectively [4].

However, there exists the problem that these tests do not distinguish immunologic memory from true infection. Current directions from the scientific community suggest that anyone with a risk factor for tuberculosis who tests positive for either the PPD or IGRA assay, should take medication. Therefore, a putative population without LTB but with acquired specific immunity against M.TB. antigens may be subjected to redundant therapy with possibly serious side-effects i.e. hepatitis. For example, the risk for HIV patients, who already receive special medication and have a fymatin positive test, to develop tuberculosis is 50% over their entire life span, which means that half of the individuals of this specific cohort will receive redundant therapy for LTB. Moreover, the side-effects of M.TB. therapy in combination with anti-retroviral therapy complicate patient status and in some cases, could be life-threatening.

Consequently, more specific tests which discern true M.TB. infection from LTBI are highly desirable and very useful since those tests [symbol] identification of individuals who will truly benefit from LTBI treatment in contrast to those who, having destroyed the bacillus with acquired immunity, retain immunological memory against M.TB. antigens.

Since current diagnosis tests (microbiology, Nucleic Acid Amplification Test) lack sufficient sensitivity to characterize tuberculosis (either pulmonary or extra pulmonary) and especially severe versions of TB (such as tuberculous meningitis, multiple organ disease, hematogenous dissemination), new high accuracy and rapid methods of M.TB. detection are of high priority and are constantly searched.

Current methods for TB diagnosis lack sensitivity, so they cannot accurately rule out TB.

The conventional microbiology test (i.e. direct smear and culture test) also lacks sensitivity for the diagnosis of pulmonary TB, with an estimated 30% of active pulmonary TB cases appearing as sputum smear and culture negative. In addition, culture-restricted results are delayed, emerging after a long time(aprox. after fifteen days).

The Nucleic Acid Amplification Test (NAAT) provides a reliable way of increasing the specificity of diagnosis (confirmation of disease) but sensitivity is too low to rule out the disease, especially in smear-negative TB. These tests, therefore, cannot be used reliably for ruling out disease especially in cases where clinical diagnosis is questionable or when clinical intervention is of high priority (5).

As a result, due to the disadvantages of the current diagnostics, in clinical practice a therapeutic trial for TBC is used as therapeutic diagnostic criterion, especially in critically ill patients, in spite of the negative diagnostic microbiology and molecular tests. As a consequence, this leads to an unnecessary treatment with increased cost and many times this is associated with serious side effects of treatment and could be life threatening. In addition, the Tb medication lasts for several months, usually 6-9 months.

### DESCRIPTION OF THE METHOD OF THE PRESENT INVENTION

The present invention solves the above described problems and for the first time provides a direct, sensitive and specific method for M.TB. detection, not only in LTB cases, but also in those cases accompanied by severe forms of tuberculosis.

The primary route of infection from M.TB. involves the lungs. Inhaled droplet nuclei avoid bronchal defenses due to their small size and [symbol] . veoli where they are engulfed by phagocytic immune cells (macrophages and dendritic cells). M. tuberculosis can also infect nonphagocytic cells in the alveolar space, including alveolar endothelial, and type 1 and type 2 epithelial cells (pneumocytes) (6). In addition, a large number of microbes is transfered via macrophages to draining lymph nodes.

Studies of the M TBC genome have led us to the discovery of a genomic region, termed region of difference 1 (RD1), that is present in all virulent M. tuberculosis and M. bovis strains but absent in the vaccine strain M. bovis BCG (7-9). The virulence of M. tuberculosis stems from RD1 products and especially the ESAT-6 and CFP-10 proteins. Region RD1 is an important research field for new vaccines, as it offers more effective and promising venues than the BCG vaccine as well as new therapies.

Recent studies have established the role of the ESX-1 secretion system and ESAT-6 protein of M. tuberculosis in facilitating macrophage infection and subsequent bacterial escape to infect other nearby cells. De Jonge et al. [10] have shown that the ESAT-6:CFP-10 complex secreted by live M. tuberculosis inside the phagosome, splits apart when tubercle bacilli are perturbed following stressful conditions created by the host. ESAT-6 lyses the phagosome creating pores, bacilli enter the cytoplasm lysing the cell membrane and then infect other host cells (6). The protein inserts itself into the lipid bilayer, causing lysis and escape of M. tuberculosis from the phagosome. More recently, it was demonstrated that ESAT-6 causes cytolysis of type 1 and type 2 pneumocytes and that ESAT-6 induced lysis contributes to bacilli dissemination to alveolar wall (6).

The intergrated experience of the inventors and their thorough knowledge of tubercle infections, not only in relation to the research field but also to clinical and laboratory practices, led to the assessment by the inventors that as the presence of ESAT-6 is linked to metabolically active M.TB., its intracellular expression should be a usefull biomarker not only for active tuberculosis but also for LTB. Therefore, we considered that this intracellular expression of ESAT-6 should have a prognostic value as regards either LTB or tuberculosis diagnosis, because it indirectly identifies, via ESAT-6, live and virulent bacilli, especially in cases that sensitivity is too low. Until now, ESAT-6 has not been ‘targeted’ by anyone as an activity marker of M.TB. infection in the manner described below by the inventors. In addition, so far, no one has thought of implementing such a method for [symbol] use of ESAT-6 is limited to studies either relating to the pathophysiology context of M.TB. infection or as a specific index of immunological memory in IGRA studies. Those approaches are in a completely different direction than that proposed by the inventors.

### SUMMARY OF THE INVENTION

The inventors consider vital the search for the presence of ESAT-6 in the interior of cells targeted by M.TB. bacilli, such as leukocytes, bronchoalveolar lavage macrophages, cells from cerebrospinal fluid, pleural effusions, pericardial effusions, synovial fluids, sperm, induced sputum, vaginal secretions and in general every biological fluid sample collected from the human body and cell suspensions from serositis effusions, mucosal eluants or solid tissues.

The present invention describes a method of indirect detection of active M.TB. inside cells via the cytoplasmic detection of ESAT-6, using either indirect or direct immunofluorescence techniques, followed by visualization and evaluation of the resut by flow cytometry

In particular, the current invention describes a method for intracellular detection using specific antibodies against RD1-M.TB. genorne products in human cells removed from the human organism.

Preferably, the intracellular detection method set forth in this invention combines the immunofluorescence technique with flow cytometry.

It is particularly advantageous that specific antibodies are conjugated directly or indirectly with a fluorochrome and, in addition, the result is evaluated by flow cytometry, wherein any fluorochrome can be used for fluorescence.

The said fluorochromes can be selected from the following: Fluorecein-5-isothiocyanate (FITC), aminomethylcoumarin Acetate (AMCA 350), 6,8-difluoro-7-hydroxycoumarin derivative (Marina Blue), Cascade Blue, Alexa fluor 405, 6,8-difluoro-7-hydroxycoumarin derivative (Pacific Blue), Alexa Fluor 430, Cascade Yellow, Alexa Fluor 488, phycoerythrin (PE), phycoerythrin Texas Red (PE-Texas Red), phycoerythrin-cyanin 5 (PE-Cy5), peridinin chlorophyll protein (PerCP), peridinin chlorophyll protein-cyanin 5.5 (PerCP-Cy5.5), phycoerythrin-cyanin 7(PE-Cy7), Rhodamine TR, allophycocyanin (APC), ALexa Fluor 647, allophycocyanin cyanin 7 (APC-Cy7), BD APC-H7, Alexa Fluor 700.

Preferably, cells to be assayed as in the current invention should preferably come from isolated pleural effusions, pericardial effusions, synovial fluids, sperm, induced sputum, vaginal secretions, in general every biological fluid sample collected from the human body and cell suspensions from serositis effusions, mucosal eluants or solid tissues, which previously had been removed from the human organism.

It is advantageous that the antibody used in the method of the present invention is a specific type of anti-ESAT-6.

It would also be advantageous to use a mixture of antibodies (including ESAT-6) recognizing more than one product of the RD1 region. Moreover, an additional stage of fluorophore-conjugated antibodies that recognize human antigen epitopes for infected cell determination can also be integrated into the whole procedure.

The present invention also describes a kit for its implementation. The said kit includes either an anti-ESAT-6 antibody or another antibody against other RD1 products of the M.TB. genome, together with additional reagents for the intracellular detection of the M.TB. bacillus in human cells that have already been removed from human organisms. It is advantageous that the above described kit comprises a combination of fluorophore-conjugated antibodies against human antigen epitopes.

In addition, the present invention discloses an automated flow cytometer, in which the whole procedure is performed automatically (smart flow cytometry). The said automated flow cytometer incorporates the kit for the direct detection of RD1 products in parallel with the evaluation of any other phenotypical parameter (sub-populations of CD4, CD8 cells), and in combination with the detection and characrerization of viruses. Preferably, the apparatus described by the present invention designates RD1 products such as ESAT-6, CFP-10, TB7.7, Ag85 etc, in cells collected from all biological fluids that have already been removed from the human organism.

Moreover, it is advantageous that along with the detection of one or more RD1 products, the apparatus described in the present invention can perform leukocyte functional assays and phenotypical analysis, i.e. it can assess the CD4/CD8 index in HIV patients from cells that have already been removed from the human organism.

The method of the present invention comprises the following stages: cell fixation, direct or indirect intracellular immunofluorescence, visualization and evaluation of the result with flow cytometry.

The method of the present invention detects active tuberculosis with the identification of a sequence of the ESAT-6 protein. It is also feasible to target other RD1 products with a view to obtaining the same result

The method of the present invention for immufluorescence, employs any anti-antibody conjugated with suitable fluorescence and can use any fluorophore, comprising any of the following currently known: Fluorecein-5- isothiocyanate (FITC), aminomethylcoumarin Acetate (AMCA 350), 6,8-difluoro-7-hydroxycoumarin derivative (Marina Blue), Cascade Blue, Alexa fluor 405, 6,8-difluoro-7-hydroxycoumarin derivative (Pacific Blue), Alexa Fluor 430, Cascade Yellow, Alexa Fluor 488, phycoerythrin (PE), phycoerythrin Texas Red (PE-Texas Red), phycoerythrin-cyanin 5 (PE-Cy5) , peridinin chlorophyll protein (PerCP), peridinin chlorophyll protein-cyanin 5.5 (PerCP-Cy5.5), phycoerythrin-cyanin 7(PE-Cy7), Rhodamine TR, allophycocyanin (APC), ALexa Fluor 647, allophycocyanin cyanin 7 (APC-Cy7), BD APC-H7, Al-exa Fluor 700.

The method of the present invention can additionally include an extracellular immunofluorescence procedure so that the cell's phenotype containing the bacilli can be identified.

The invention also describes the development and use of a kit (i.e. a product in a special package that includes reagents and necessary instructions for the assay procedure). In addition, the above kit comprises one or more antibodies against the ESAT-6 protein since it is our intention to detect it. If these antibodies are conjugated, the anti-antibody is not necessary; in case the antibodies are not conjugated, the kit will further comprise a second antibody (anti-antibody) conjugated to a fluorophore or biotin.

### EXAMPLE

An example of the implementation of the current invention is the following: we choose to look for the presence of ESAT-6 in the peripheral blood of a patient with active disease. In addition, a normal donor who is negative for any manifestation of disease in all clinical assays that assess tuberculosis is used as a negative control.

### Fixation of Blood Cells

Approximately 200 ul of peripheral blood are spun down and fixed with 4% Para-formaldehyde (PFA) at 4° C. for 30 min. PFA preserves the physical characteristics of cells during analysis with flow cytometry. Moreover, PFA allows the operator to perform future staining procedures of either an extracellular or intracellular nature and to dispatch fixed cells to laboratories that have suitable apparatus.

### Intracellular Immunofluorescence Staining (Indirect)

In this example, indirect immnofluorescence is used, because it is is less costly than having all the antibodies directly conjugated.

A. A fraction of the cells is spun down, resuspended, and incubated for 30 min in 100 pl Phosphate Buffered Saline (PBS)-0.1% saponin (medium A). Saponin creates pores at the cellular membrane making intracellular access feasible. Therefore, an antibody namely, one intracellulary targeting the ESAT-6 antigen epitope (11G4) can be linked to its target epitope. The cells are spun down again, the supernatant is discarded, the pellet resuspended in 50 μl of medium A and incubated with titered amounts of the antibody against the ESAT-6 epitope, such as for example, 11G4 which targets the EQQWNFAGIEAAA epitope. The cells are incubated for 30 min at 4° C.

The cells are then washed with 2 ml PBS containing 0.1% saponin and 2% Fetal Calf Serum (FCS) (wash buffer-WB) and the supernatant is discarded.

B. The cell pellet is resuspended and a new incubation with a tittered amount of a polyclonal fluorophore-conjugated antibody against immunoglobulins from the animal from which the first antibody (fluorophore conjugated anti-antibody) was developed, follows.

Any fluorophore can be used. The following are the most commonly known fluorophores; they are listed here indicatively and are not limited to: Fluorecein-5-isothiocyanate (FITC), aminomethylcoumarin Acetate (AMCA 350), 6,8-difluoro-7-hydroxycoumarin derivative (Marina Blue), Cascade Blue, Alexa fluor 405, 6,8-difluoro-7-hydroxycoumarin derivative (Pacific Blue), Alexa Fluor 430, Cascade Yellow, Alexa Fluor 488, phycoerythrin (PE), phycoerythrin Texas Red (PE-Texas Red) [symbol] peridinin chlorophyll protein-cyanin 5.5 (PerCP-Cy5.5), phycoerythrin-cyanin 7(PE-Cy7), Rhodamine TR, allophycocyanin (APC), ALexa Fluor 647, allophycocyanin cyanin 7 (APC-Cy7), BD APC-H7, Alexa Fluor 700.

Following a 30 min incubation at 4 ° C., the cells are washed with 2 ml WB. The supernatant is discarded and the cell pellet is resuspended in 50 μl of PBS. If it is necessary to perform a leukocyte study to determine presence of ESAT-6, the procedure goes on to next step. Alternatively, the procedure proceeds to the step where the cells are harvested.

### Optional Step of Leukocyte Staining

If we wish to determine the phenotype of the cell that contains the ESAT-6 protein, a classic immunofluorescence assay can be performed and the cells incubated with an antibody i.e. against granulocytes (anti CD13) which is directly conjugated with a different fluorochrome than that used for ESAT-6 assession. Following a 30 min incubation at 4° C., the samples are washed with 2 ml PBS containing 2% FCS. The supernatant is discarded, the cells are resuspended in PBS and are ready for acquisition in a Flow Cytometer.

### Acquisition and Evaluation of the Results

The samples are acquired in a flow cytometry apparatus. The data analysis is performed using suitable software where we use region combinations based on scatter standards (size, complexity) and/or expression of leukocyte antigens. The analysis focuses on detection of the intracellular presence of the EQQWNFAGIEAAA sequence of ESAT-6, identified from the 11G4 clone, in certain cell populations (as depicted in FIG. 1D). In this particular example, the presence of the antigenic epitope has been analyzed in the interior of granulocytes based on their scatter characteristics.

### DESCRIPTION OF FIGURES

The invention is presented with the following figures:

FIGS. 1A, 1B, 1C show the intracellular detection of epitope EQQWNFAGIEAAA belonging to the ESAT-6 protein, in the granulocytes of an active tuberculosis patient with flow cytometry. The antibody is: anti-Mycobacterium tuberculosis, ESAT-6 Monoclonal Antibody, clone 11G4 (Thermo SCIENTIFIC). This antibody is recommended by manufacture and the literature [symbol] (WB) use and NOT for immunofluorescence assays (Product Data Sheet). In contrast to current practice, the inventors chose to use this antibody since their laboratory research experience and clinical knowledge, have led them to consider it appropriate to detect latent or active tuberculosis.

We can analyze different regions, so that we can focus on any particular cell population.

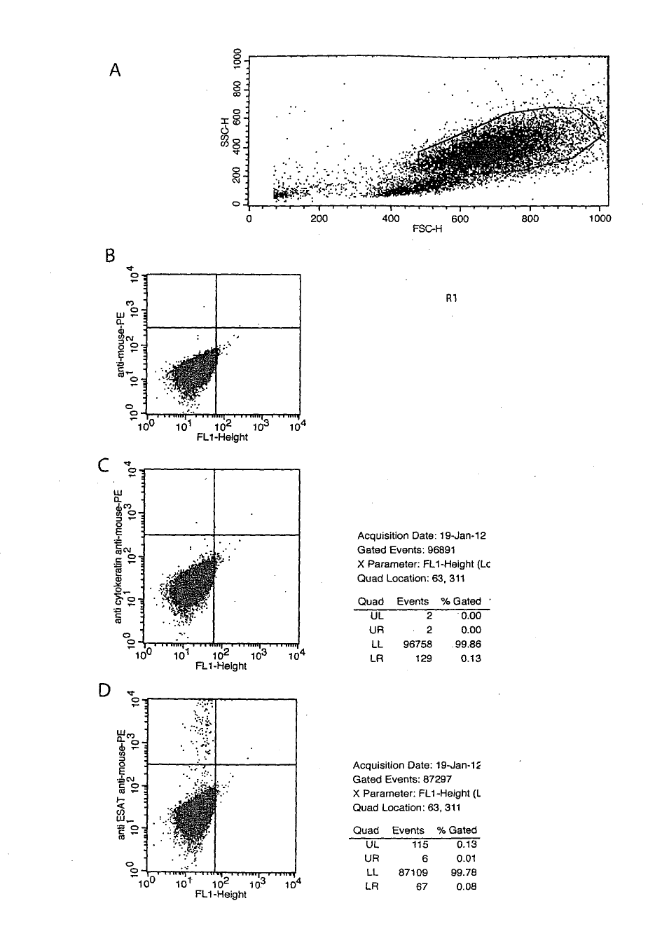

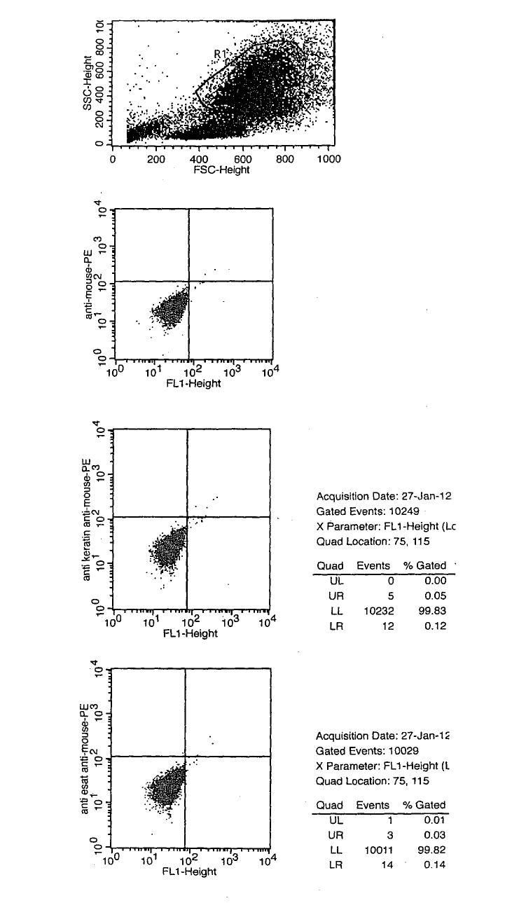

In FIG. 1A, a region has been drawn, which according to scatter characteristics corresponds to granulocytes and so we focus our analysis on this region.

In FIG. 1B, non-specific staining of cells using only the second antibody, without presence of the first one, 11G4, is shown.

In FIG. 1C, cells have been incubated with an antibody that detects the presence of keratin, a molecule not expressed in blood leukocytes. It is considered a negative control. From this figure, we can infer the correctness of the described technique, since this non-specific antibody which detects keratin, a protein not expressed in leukocytes, cannot stain leukocytes. In particular, the absence of dots in the upper left quadrant of FIG. 1C, shows that there is no cell positive for keratin. Consequently, the described technique has reliability safeguards as regards extraction of false positive results.

FIG. 1D, shows cells in which the 11G4 target sequence is detected. Specifically, as resulted from the analysis of the cytometer (FACScan, Becton Dickinson) data using the CellQuest analysis program, 0.13% of gated granulocytes express the ESAT-6 protein. In the upper left quadrant of FIG. 1D, dots, assessing ESAT-6 positive granulocytes, are shown. We therefore, conclude the specificity of 11G4 detection.

FIGS. 2A, 2B, 2C and 2D depict the same parameters, but the cells are from a healthy donor, free of tuberculosis.

FIG. 2A depicts a region including granulocytes, while FIG. 2B shows non specific staining with the sole presence of a second antibody.

In FIG. 2C, the absence of keratin positive cells is shown—i.e. the extraction of false positive results is successful.

In FIG. 2D, absence of the ESAT-6 antigen detection in the granulocytes of a healthy donor is shown.

### Advantages of the Method Described in the Current Invention

An immunophenotypic method of intracellular detection of M.TB. is dercibed. The main goal is the direct detection of M.TB. which causes either infection or latent infection. In the present invention, the presence of M.TB. infection is detected using an antibody against the intracellular presence of whatever product of the RD1 region, which substantiates the presence of metabolically active bacilli.

As reported above, application of current diagnostic methods results in that at least 30% of cases escape diagnosis. According, to our current data the sensitivity of the novel method of the present invention is greater than that yielded by methodologies applied so far. This greater sensitivity, as well as the fact that RD1 products are an index of active infection, strengthen the clinical value of the present invention, substantially contributing to the identification and selection of infected individuals who should undergo therapy.

In contrast to microbiological and molecular tests whichrequire the presence of 5000-10000 bacilli per ml of a sputum sample, the described method allows for ESAT-6 expression assessment in each and every cell, which results in higher accuracy. In addition, we note that 30% of sputum smears are found to be negative for active and contagious tuberculosis, when any of the currently known diagnostic methods is applied.

Furthermore, with the present invention we can measure a large number of cells (even a million), in order to indirectly assess and detect M.TB.

As reported above, the current literature shows that there is no reliable method of distinghuishing between infection detection and immunological memory, i.e. discriminating immunological memory without the existence of bacilli from infection in the presence of bacilli.

The immunophenotypic method of RD1 product intracellular detection is very accurate since the antibody used is monoclonal, which means that itr ecognizes only one epitope. In addition, a negative control is included. which substantiates the specificity. Furthermore, itdoes not cross-react with either humoral infectious intermediates or activation agents arising from previous stages of infection (memory). The retrospective analysis of results is also feasible, on demand.

In addition to reliably characterizing negative controls and positive samples, our method facilitates quantitative assessment of infectious burden since the percentage of infected cells (usually [symbol] of the total cells. The percentage of granulocytes that stained positive for RD1 protein products, such as ESAT-6, represents the activity of infection.

From a technical point of view, the method of intracellular detection of antigen, is simple, fast, reproducible and has low cost. The result is delivered after 90 minutes, following either blood draw or other material collection.

Remote sample transport is safe, without special precautions, as is usualy the case with infectious material transportation, since sample virulence is neutralized with fixation, while the possibility of detecting already inactivated, via fixation, viruses such as HIV, andhepatitis also remains.

The only necessary equipment for implementating the proposed method is a flow cytometry apparatus in combination with basic laboratory equipment (centrifuge, etc).

The protocol does not demand specialized personel for its implementation since it is simple in its use.

### BIBLIOGRAPHY

1. Newton, S. M., Brent, A. J., Anderson, S., Whittaker, E., and Kampmann, B. 2008. Paediatric tuberculosis. Lancet Infect Dis 8:498-510.
2. WHO. Global Tuberculosis Control 2009: Epidemiology, strategy,financing. Geneva, WHO Report, 2009.
3. Pai, M., Zwerling, A, and Menzies D. 2008. Systematic Review: T-Cell-based Assays for the Diagnosis of Latent Tuberculosis Infection: An Update. Ann Intern Med 149:177-184.
4. Sester, M., Sotgiu, G., Lange, C., Giehl C., Girardi, E., Migliori, G. B., Bossink A, Dheda, K., Diel, R., Dominguez, J., Lipman, M., Nemeth, J., Ravn, P., Winkler, S., Huitric, E., Sandgren, A., and Manissero, D. 2011. Interferon-γ release assays for the diagnosis of active tuberculosis: a systematic review and meta-analysis. Eur Respir J 37:100-111.
5. Dinnes, J., Deeks, J., Kunst, H., Gibson, A., Cummins, E., Waugh, N., Drobniewski, F., and Lalvani, A. 2007. A systematic review of rapid diagnostic tests for the detection of tuberculosis infection. Health Technol Assess 11:1-196.
6. Kinhikar, A. G., Verma, I., Chandra, D., Singh, K. K., Weldingh, K., Andersen, P., Hsu, T., Jacobs, W. R., Jr., and Laal, S. 2010. Potential role for ESAT6 in dissemination of M. tuberculosis via human lung epithelial cells. Mol Microbiol 75:92-106.
7. Cole, S. T., Brosch, R., Parkhill, J., Gamier, T., Churcher, C., Harris, D., Gordon, S. V., Eiglmeier, K., Gas, S., Barry, C. E., 3rd, et al. 1998. Deciphering the biology of Mycobacterium tuberculosis from the complete genome sequence. Nature 393:537-544.
8. Mahairas, G. G., Sabo, P. J., Hickey, M. J., Singh, D. C., and Stover, C. K. 1996. Molecular analysis of genetic differences between Mycobacterium bovis BCG and virulent M. bovis. J Bacteriol 178:1274-1282.
9. Behr, M. A., Wilson, M. A., Gill, W. P., Salamon, H., Schoolnik, G. K., Rane, S., and Small, P. M. 1999. Comparative genomics of BCG vaccines by whole-genome DNA microarray. Science 284:1520-1523.
10. de Jonge, M. I., Pehau-Arnaudet, G., Fretz, M. M., Romain, F., Bottai, D., Brodin, P., Honore, N., Marchal, G., Jiskoot, W., England, P., et al. 2007. ESAT-6 from Mycobacterium tubrtculosis [symbol] chaperone CFP-10 under acidic conditions and exhibits membrane-lysing activity. J Bacteriol 189:6028-6034.

1. Method for intracellular detection of Mycobacterium tuberculosis with the use of specific antibodies against RD1 products of the Mycobacterium tuberculosis genome, in human cells which had been removed from human organisms.

2. The method according to claim 1 where immunofluorescence is used in combination with flow cytometry.

3. The method according to claim 2 where specific antibodies are either directly or indirectly conjugated with a fluorophore molecule, the result is evaluated with flow cytometry and any fluorochrome can be used for fluororescence.

4. The method according to claim 3 where fluorochromes can be any of the following: Fluorecein-5- isothiocyanate (FITC), aminomethylcoumarin Acetate (AMCA 350), 6,8-difluoro-7-hydroxycoumarin derivative (Marina Blue), Cascade Blue, Alexa fluor 405, 6,8-difluoro-7-hydroxycoumarin derivative (Pacific Blue), Alexa Fluor 430, Cascade Yellow, Alexa Fluor 488, phycoerythrin (PE), phycoerythrin Texas Red (PE-Texas Red), phycoerythrin-cyanin 5 (PE-Cy5), peridinin chlorophyll protein (PerCP), peridinin chlorophyll protein-cyanin 5.5 (PerCP-Cy5.5), phycoerythrin-cyanin 7(PE-Cy7), Rhodamine TR, allophycocyanin (APC), ALexa Fluor 647, allophycocyanin cyanin 7 (APC-Cy7), BD APC-H7, Alexa Fluor 700.

5. The method according to claim 1, wherein cells are collected from any of the following: peripheral blood, cerebrospinal fluid, pleural effusions, pericardic effusions, ascitic fluids, synovial fluid, sperm, sputum, vaginal secretions, and, in general from every biological fluid sample collected from the human body as well as from cell suspensions containing cells from serositis effusions, any mucosal eluant or solid tissues which previously had been removed from the human organism.

6. The method according to claim 1, wherein the antibody is specific for the ESAT-6 molecule (anti-ESAT-6).

7. The method according to claim 1 where a mixture of antibodies is used for more than one RD1 region products, including ESAT-6.

8. The method according to claim 1 with an extra stage of using fluorescent antibodies recognizing human epitopes for the phenotypic identification of infected cells.

9. A kit comprising an anti-ESAT-6 antibody or antibodies against other RD1 products of the M.TB. genome, together with additional reagents for the intracellular detection of the M.TB. bacillus in human cells that have already been removed from human organisms.

10. The kit, according to claim 9 which also comprises a combination of other fluorescent antibodies specific for human molecules.

11. An automated flow cytometer, which automatically performs the whole procedure (smart flow cytometry) and integrates the kit of claim 9 for the direct detection of RD1 products, in parallel with evaluation of any other phenotypical parameter of the cells (subpopulations of CD4, CD8) that have already been removed from the human organism, and in combination with any other parameter that can be evaluated and also in parallel it performs detection and characrerization of viruses.

12. The kit according to claim 9 with RD1 products such as ESAT-6, CFD-10, TB 7.7, AG85, etc. collected from biological fluids that have already been removed from the human organism.

13. The kit according to claim 9, wherein together with the detection of one or more RD1 products, it performs leukocyte functional assays and phenotypical analysis, as for example it can assess the CD4/CD8 index in HIV patients from cells that have already been removed from the human organism.
